(19) United States
(12) Patent Application Publication
(10) Pub. No.: US 2016/0303264 A1
(43) Pub. Date: Oct. 20, 2016

(54) LIPOSOMES USEFUL FOR NON-INVASIVE IMAGING AND DRUG DELIVERY

(71) Applicant: MERRIMACK PHARMACEUTICALS, INC., Cambridge, MA (US)

(72) Inventors: BART S. HENDRIKS, BELMONT, MA (US); HELEN LEE, CAMBRIDGE, MA (US); VICTOR MOYO, RINGOES, NJ (US); THOMAS WICKHAM, GROTON, MA (US)

(73) Assignee: MERRIMACK PHARMACEUTICALS, INC., Cambridge, MA (US)

(21) Appl. No.: 15/031,643

(22) PCT Filed: Oct. 23, 2014
(86) PCT No.: PCT/US14/62007

§ 371 Date: Apr. 22, 2016

Related U.S. Application Data
(60) Provisional application No. 61/894,854, filed on Oct. 23, 2013.

(51) Int. Cl. A61K 51/10; A61K 9/127; A61K 9/00; A61K 51/12; A61K 31/704

(52) U.S. Cl. CPC A61K 51/1051; A61K 51/1063; A61K 51/1234; A61K 31/704; A61K 9/0019; A61K 9/127

(57) ABSTRACT

The present invention relates to liposomes useful for diagnosis and/or therapy of a target site, such as cancerous tissue. The compositions and methods disclosed herein find particular use in diagnosing and imaging cancerous tissue. The present invention provides a new diagnostic tool for the utilization of positron emission tomography (PET) computed tomography imaging technique.

### CROSS-REFERENCE TO RELATED APPLICATIONS

This application is a 35 U.S.C. §371(c) United States national phase application of PCT/US2014/062007, filed Oct. 23, 2014, which claims the benefit of and priority to U.S. Provisional Patent Application No. 61/894,854, filed Oct. 23, 2013. The entire contents of the foregoing applications are incorporated herein by reference in their entireties.

### BACKGROUND

Liposomes have proved a valuable tool for delivering various pharmacologically active molecules, such as anti-neoplastic agents, to cells, organs, or tumors. Liposome delivery has been shown to improve the pharmacokinetic profile and widen the therapeutic index of certain anticancer drugs, especially the anthracycline class. Improved efficacy is in part a result of passive targeting to tumor sites based on the enhanced permeability and retention (EPR) effect. To fully exploit this process, drug carriers should be engineered to retain drug while circulating, thereby preventing premature drug release before accumulating in the tumor but still allowing for release of drug once in the vicinity of the tumor. Antibody-targeted nanoparticles, such as immunoliposomes comprising external antibodies or antibody fragments that immunospecifically bind, for example, HER2 or epidermal growth factor receptor, represent another strategy for more efficient drug delivery to tumor cells.

It has been found, however, that deposition of liposomal drugs into tumors varies and tumors that exhibit higher liposomal drug deposition will have improved clinical outcomes. Liposomal drugs have been shown to accumulate in tumors via a mechanism termed the enhanced permeability and retention (EPR) effect whereby liposomes preferentially escape from the bloodstream into the tumor interstitium via leaky tumor vasculature and then become trapped in the tumor by virtue of their large size and the reduced levels of functional lymphatics in the tumor. However, the degree to which liposomal particles can deposit into tumors has been shown to be highly variable in both preclinical tumor models and in clinical studies.

Compositions and non-invasive methods allowing the determination of whether or not a liposomally-delivered therapeutic agent is suitable for use in a patient (e.g., to predict clinical outcomes of targeted and untargeted liposomal cancer therapeutics) are therefore needed.

### SUMMARY

Provided herein are liposomal imaging agents that can be used to predict low or high deposition of liposomal drugs in lesions (e.g., localized pathology such as cancers, malignant or benign tumors, and sites of inflammation or infection) in a patient, and ultimately which patients will benefit from a particular liposomal drug, as well as methods for their use. Also disclosed herein are methods for non-invasive imaging, and more particularly, for non-invasive imaging for use in predicting the utility of liposomal therapeutics. Such methods are useful in imaging cancer or another disease (e.g., a localized infectious or inflammatory disease), and/or for drug delivery to a target site, e.g., tumor tissue. In some embodiments, the method further comprises treating a patient, e.g., a patient having an infection, a localized inflammatory condition, or a cancerous tumor. For example, a preparation of liposomes may contain a chemotherapeutic agent, such as a taxane, a topoisomerase inhibitor (e.g., irinotecan or topotecan), or an anthracycline (e.g., doxorubicin), in the liposomal interior space and liposomes comprised by the preparation may be loaded with a radiolabel suitable for PET imaging, such as ⁶⁴Cu, thus allowing for imaging and treatment to result from the same administration of the liposomal preparation.

In a first aspect, disclosed herein is a method of preparing a patient for PET imaging of a lesion in the patient, the method comprising administering to the patient an injection comprising a dose of a preparation of ⁶⁴Cu-loaded liposomal doxorubicin, the liposomes comprised by the preparation having an average diameter of 75-110 nm, wherein the dose comprises 3-5 mg/m² doxorubicin and is formulated to deliver 10.8 (+/−15%, optionally +/−10%) millicuries (mCi) of ⁶⁴Cu when administered to the patient. In one embodiment, the lesion is a benign tumor or a malignant tumor, optionally a brain tumor. In another embodiment, the ⁶⁴Cu-loaded liposomes are immunoliposomes. In one embodiment, the immunoliposomes are HER2-targeted immunoliposomes. In another embodiment, the immunoliposomes are EphA2-targeted immunoliposomes. In some embodiments the liposomes comprise a gradient-loadable chelator. In one embodiment, the chelator is 4-DEAP-ATSC.

In a second aspect, disclosed herein is a method of imaging a lesion in a patient, the method comprising administering to the patient an injection comprising a preparation of ⁶⁴Cu-loaded liposomal doxorubicin, the liposomes comprised by the preparation having an average diameter of 75-110 nm, at a dose of 3-5 mg/m² doxorubicin; then within 48 hours following the injection, obtaining a PET scan of a region of the patient, the region comprising the location of the lesion. In one embodiment, the lesion is a site of inflammation, a site of infection, a benign tumor or a malignant tumor, optionally a malignant brain tumor. In another embodiment, the dose of liposomal doxorubicin is formulated to deliver to the patient, when administered, 10.8 (+/−15%, optionally +/−10%) mCi of ⁶⁴Cu. In another embodiment, the PET scan is obtained within 24 hours, within 12 hours, within six hours, within 3 hours, within 2 hours, or within 1 hour following the injection.

In a third aspect, disclosed herein is a method of imaging a lesion in a patient, the method comprising: (a) administering to the patient an injection comprising a preparation of 64Cu-loaded liposomes, the injection administered at a dose of 10.8 mCi of ⁶⁴Cu (+/−15%); and (b) within 48 hours following the injection, obtaining a PET scan of a region of the patient, the region comprising the location of the lesion. In one embodiment, the preparation comprises liposomes with an average diameter of 75-110 nm. In another embodiment, the PET scan is obtained within 3 hours following the injection. In one embodiment, the within 3 hours is within 2 hours or within 1 hour. In one embodiment, the ⁶⁴Cu-loaded liposomes are immunoliposomes.

In one embodiment, the immunoliposomes are HER2-targeted immunoliposomes. In another embodiment, the immunoliposomes are EphA2-targeted immunoliposomes. In some embodiments the liposomes comprise a gradient-loadable chelator. In one embodiment, the chelator is 4-DEAP-ATSC. In one embodiment, the liposomes further comprise a chemotherapeutic agent. In some embodiments the chemotherapeutic agent is doxorubicin or irinotecan or a taxane. In one embodiment, the lesion is a brain tumor.

In a fourth aspect, disclosed herein is a method of treating and imaging a patient, the method comprising: (a) administering to the patient a first injection comprising immunoliposomal doxorubicin that does not comprise detectable levels of ⁶⁴Cu, the injection administered at a dose of at least 25, at least 30, at least 35, at least 40, or at least 45, or 50 mg/m² of doxorubicin; (b) at between one and 6 hours following the first injection, administering to the patient a second injection comprising ⁶⁴Cu-loaded immunoliposomal doxorubicin, the doxorubicin comprised by the second injection consisting of a dose of at least 3, at least 4, at least 5, at least 6, or 7 mg/m² of doxorubicin, said dose comprising at 10.8 mCi of ⁶⁴Cu+/−15%; and (c) obtaining at least two PET/CT scans of a region of pathology in the patient, wherein each scan is obtained at a different time point, and wherein time elapsed from the injection of (a) until a final scan of the at least two scans is obtained is no more than three days. In one embodiment, the immunoliposomal doxorubicin is HER2-targeted. In another embodiment, the immunoliposomal doxorubicin is EphA2-targeted.

In a fifth aspect, disclosed herein are compositions comprising ⁶⁴Cu-loaded liposomes containing doxorubicin, such compositions being useful in practicing the methods disclosed herein. In one embodiment, the liposomes are HER2-targeted liposomes. In another embodiment, the liposomes are EphA2-targeted immunoliposomes. In some embodiments, the composition is adapted for administration to a human patient at a dose of at least 0.028, at least 3, at least 4, at least 5, at least 6, or 7 mg/m2 of doxorubicin. In one embodiment, the liposomes comprise a gradient-loadable chelator. In one embodiment, the chelator is 4-DEAP-ATSC. In another embodiment, the composition comprises about 5, about 7, about 10, about 10.8, about 12, or about 15 mCi of ⁶⁴Cu. In one embodiment, the liposomes comprise hydrogenated soy phosphatidylcholine (HSPC), cholesterol, and poly(ethylene glycol) (PEG)-derivatized distearoylphosphatidylethanolamine (PEG-DSPE) at a 3:1:0.05 molar ratio. In one embodiment, the poly(ethylene glycol) of the PEG-DSPE has a molecular weight of about 2000.

Other features and advantages will be apparent from the detailed description, and from the claims.

### BRIEF DESCRIPTION OF THE DRAWINGS

FIG. 1 is a drawing illustrating the use of a chelator to load ⁶⁴Cu into a transmembrane gradient-containing liposome that contains an entrapped drug.

FIG. 2 is three graphs demonstrating the in vitro stability of ⁶⁴Cu-Liposomes incubated at 37° C. for up to 48 hours in human plasma. FIGS. 2A and 2B show the retention of ⁶⁴Cu in Liposome B as shown by size exclusion chromatography. FIG. 2C shows the amount of ⁶⁴Cu retained within Liposome B.

FIG. 3 is two graphs showing pharmacokinetics and in vivo stability of ⁶⁴Cu-Liposome B in non-tumor-bearing CD-1 mice. FIG. 3A shows the results of measurement of both ⁶⁴Cu and doxorubicin in plasma samples. FIG. 3B shows the stability of the ⁶⁴Cu labeled liposomes by comparison with doxorubicin.

FIG. 4 is a graph showing biodistribution of ⁶⁴Cu-Liposome B in BT474-M3 mammary fat pad xenograft models, demonstrated by the measurement of both doxorubicin and ⁶⁴Cu.

FIG. 5 is two graphs showing that liposome targeting has no effect on the total tumor deposition of Liposome B and its untargeted counterpart (FIG. 5A), but rather, increases the liposome uptake by tumor cells within the tumors (FIG. 5B).

FIG. 6 is a graph showing tumor deposition of Liposome B in mouse xenograft models with tumors expressing various levels of HER2. Tumor depositions of Liposome B were found to vary with no correlation with HER2 expression in the tumors. X axis is labeled with the names of the cell lines used to generate the various xenografts using which the data were obtained.

FIG. 7 is three graphs showing the in vivo stability of Liposome A (7A), Liposome B (7B), and Liposome C (7C) after injection into CD-1 mice.

FIG. 8 is three images created with aligned PET/CT images overlaying x-ray CT images (PET/x-ray overlay) of a mouse bearing a BT474-M3 mammary tumor following tail vein injection of ⁶⁴Cu-Liposome B. PET/CT images were taken at 5 minutes, 5 hours, and 20 hours. The tumors are indicated with arrowheads. Voxel intensities at each time point are decay-corrected to the time of injection.

FIG. 9 is a set of images of the liver region of a human patient obtained via x-ray CT scan (top), PET/CT scan (center) and PET/x-ray colorized overlay (bottom) taken at 33 minutes post-injection (left) and 19 hr post-injection (right). Tumors are indicated with large arrows. (ROI=region of interest noted by radiologist)

FIG. 10 is a set of images of the spinal region of a human patient obtained via x-ray CT scan (top left), PET/CT scan (center left) and colorized PET/x-ray overlay (bottom left) taken at 33 minutes post-injection. The larger image on the right shows a corresponding PET/CT scan of the same patient 19 hr post-injection. A bone lesion in the spine is indicated with an arrow and the region of this lesion is indicated on each image by a red outline.

FIG. 11 is a set of images of the cranial region (coronal section) of a human patient via x-ray CT scan (top left), PET/CT scan (center left) and colorized PET/x-ray overlay (bottom left) taken at 33 minutes post-injection. The larger image on the right shows a corresponding colorized PET/x-ray overlay image of the same patient in which the PET scan was taken 19 hr post-injection. A tumor in the brain is indicated by the large arrow and the region of this tumor is indicated on each image by a red outline. (ROI=region of interest noted by radiologist)

FIG. 12 is a graph showing examples of ⁶⁴Cu-liposome deposition kinetics in 5 lesions in a single patient within 48 hours post-injection of the liposome. PET/CT images were acquired at 0.7, 24, and 47 hours post-injection.

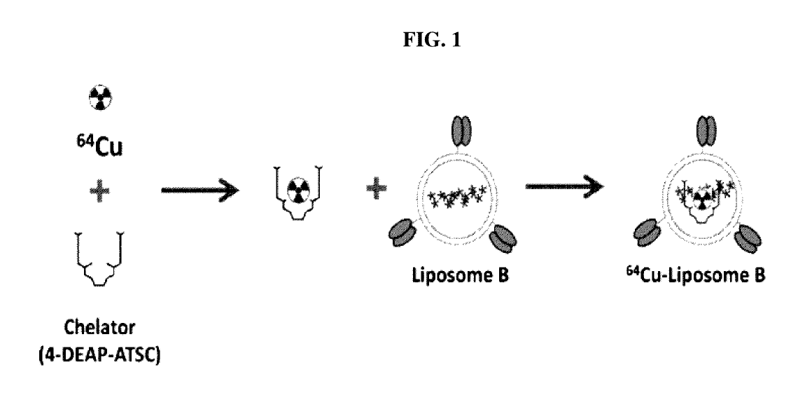

### DETAILED DESCRIPTION

The present invention provides compositions and methods for non-invasive imaging, and more particularly, non-invasive imaging for liposomal therapeutics, as well as methods of treating patients comprising the use of such methods for non-invasive imaging prior to administration of liposomal therapeutics.

The invention is based, at least in part, on the discovery that diacetyl 4,4′bis(3-(N,N-diethylamino)propyl)thiosemicarbazone (4-DEAP-ATSC) is useful as a non-invasive imaging reagent for determining whether a subject is a candidate for treatment with a liposomal therapeutic, as well as for monitoring treatment of a subject with a liposomal therapeutic.

### I. Definitions

Unless specifically stated or obvious from context, as used herein, the term “about” is understood as within a range of normal tolerance in the art, for example within 2 standard deviations of the mean. “About” can be understood as within 15%, 14%, 13%, 12%, 11%, 10%, 9%, 8%, 7%, 6%, 5%, 4%, 3%, 2%, 1%, 0.5%, 0.1%, 0.05%, or 0.01% of the stated value.

By “Liposome A” is meant a ⁶⁴Cu-loaded liposome that does not contain any drug.

By “Liposome B” is meant ⁶⁴Cu-loaded, HER2-targeted liposomal doxorubicin. Exemplary methods of preparation, dosage and administration of Liposome B may be found, e.g., in co-pending Patent Publication No. WO/2012/078695.

By “Liposome C” is meant ⁶⁴Cu-loaded irinotecan sucrosofate liposome injection. Liposome C can be prepared in accordance with U.S. Pat. No. 8,147,867.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the term “subject” or “patient” is a human patient.

By “mGy” is meant milligray, which is a measure of an absorbed dose of ionizing radiation. A Gy is defined as the absorption of one joule of radiation energy by one kilogram of matter.

By “mBq” is meant megabecquerel, which is a measure of radioactivity. One Bq is defined as the activity of a quantity of radioactive material in which one nucleus decays per second.

By “doxorubicin equivalent” is meant, in the case of liposomal doxorubicin, the total mass of doxorubicin in each dose. That is, the dosage of liposomal doxorubicin is determined based on the amount of doxorubicin in a particular volume of liposome preparation.

A substance “loaded liposomal” drug or preparation (e.g., ⁶⁴Cu-loaded liposomal doxorubicin), or substance “loaded liposomes” refer to a liposomal preparation in which the substance is entrapped within liposomes comprised by the preparation or to liposomes comprising the substance.

By “lesion,” as used herein, is meant a region in an organ or tissue that has suffered damage through injury or disease, such as a tumor (benign or malignant) or localized sites of inflammation or infection.

By “EphA2” is meant ephrin type-A receptor 2. Eph receptors are a unique family of receptor tyrosine kinases that play critical roles in embryonic patterning, neuronal targeting, and vascular development during normal embryogenesis. Eph receptor tyrosine kinases and their ligands, the ephrins, are also frequently overexpressed in a variety of cancers and tumor cell lines. EphA2 is overexpressed in, e.g., breast, prostate, lung, and colon cancers.

### II. Liposomal Imaging and Drug Delivery Agents

Disclosed herein are liposomal imaging and drug delivery agents having at least two components: (1) A liposome, which will be suspended or solubilized in a liquid medium (such as a buffer or other pharmaceutically acceptable carrier); (2) a chelator moiety capable of chelating a metal ion; and optionally (3) a metal ion suitable for imaging or otherwise assessing the in vitro or in vivo uptake of the liposomal imaging agent into cells, organs, or tumors. In some embodiments, the metal ion has a valency of 2 or 3 or 4. In exemplary embodiments, the metal ion has a valency of 2. Exemplary liposomal imaging agents are described in PCT/US13/37033.

### Liposomes

The liposomes of the liposomal imaging agents disclosed herein can be any liposome known or later discovered in the art. In certain embodiments, the liposome comprises hydrogenated soy phosphatidylcholine (HSPC), cholesterol, and poly(ethylene glycol) (PEG) (Mol. weight 2000)-derivatized distearoylphosphatidylethanolamine (PEG-DSPE) (3:1:0.05 molar ratio).

In other embodiments, the liposome comprises poly(ethylene glycol)-derivatized phosphatidylethanolamines such as 1,2-distearoyl-sn-glycero-3-phosphatidyl ethanolamine-N-[methoxy(poly(ethylene glycol))]; 1,2-dipalmitoyl-sn-glycero-3-phosphatidyl ethanolamine-N-[methoxy(poly(ethylene glycol))]; 1,2-dimyristoyl-sn-glycero-3-phosphatidyl ethanolamine-N-[methoxy(poly(ethylene glycol))]; or 1,2-dioleoyl-sn-glycero-3-phosphatidyl ethanolamine-N-[methoxy(poly(ethylene glycol))]. In certain embodiments, the molecular weight of PEG is 750, 1000, 1500, 2000, 3000, 3500, or 5000.

In certain embodiments the liposome comprises poly(ethylene glycol)-derivatized diacyl glycerols such as such as 1,2-distearoyl-glyceryl-[methoxy(poly(ethylene glycol))]; 1,2-dimyristoyl-glyceryl-[methoxy(poly(ethylene glycol))]; 1,2-dipalmitoyl-glyceryl-[methoxy(poly(ethylene glycol))]; or 1,2-dioleoyl-glyceryl-[methoxy(poly(ethylene glycol))]. In certain embodiments, the molecular weight of PEG is 750, 1000, 1500, 2000, 3000, 3500, or 5000.

In other embodiments the liposome comprises 1,2-dioctadecyl glycero-N-[methoxy(poly(ethylene glycol))]; dihexadecyl glycero-N-[methoxy(poly(ethylene glycol))]; or ditetradecyl glycero-N-[methoxy(poly(ethylene glycol))]. In certain embodiments, the molecular weight of PEG is 750, 1000, 1500, 2000, 3000, 3500, or 5000.

In various embodiments the liposome comprises PEG-ceramides, such as N-octdecanoyl-sphingosine-1-{succinoyl[methoxy(poly(ethylene glycol))]}; N-tetradecanoyl-sphingosine-1-{succinoyl[methoxy(poly(ethylene glycol))]}; N-hexadecanoyl-sphingosine-1-{succinoyl[methoxy(poly(ethylene glycol))]}; N-octdecanoyl-sphingosine-1-[methoxy(poly(ethylene glycol))]; N-tetradecanoyl-sphingosine-1-[methoxy(poly(ethylene glycol))]; or N-hexadecanoyl-sphingosine-1-[methoxy(poly(ethylene glycol))]. In certain embodiments the molecular weight of PEG is 750, 1000, 1500, 2000, 3000, 3500, or 5000.

Additional examples of suitable nanoparticle or liposome forming lipids that may be used in the compositions or methods include, but are not limited to, the following: phosphatidylcholines such as diacyl-phosphatidylcholine, dialkylphosphatidylcholine, 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine; phosphatidylethanolamines such as 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl-phosphatidylethanolamine, 1-stearoyl-2-oleoyl-phosphatidylethanolamine and N-succinyl-dioleoyl-phosphatidylethanolamine; phosphatidylserines such as 1,2-dioleoyl-phosphatidylserine, 1,2-dipalmitoyl-phosphatidylserine, 1,2-dimyristoyl-phosphatidylserine, 1,2-distearoyl-phosphatidylserine, 1-oleoyl-2-palmitoyl-phosphatidylserine, 1-oleoyl-2-stearoyl-phosphatidylserine, 1-palmitoyl-2-oleoyl-phosphatidylserine and 1-stearoyl-2-oleoyl-phosphatidylserine; phosphatidylglycerols such as 1,2-dioleoyl-phosphatidylglycerol, 1,2-dipalmitoyl-phosphatidylglycerol, 1,2-dimyristoyl-phosphatidylglycerol, 1,2-distearoyl-phosphatidylglycerol, 1-oleoyl-2-palmitoyl-phosphatidylglycerol, 1-oleoyl-2-stearoyl-phosphatidylglycerol, 1-palmitoyl-2-oleoyl-phosphatidylglycerol and 1-stearoyl-2-oleoyl-phosphatidylglycerol; pegylated lipids (lipids comprising polyethylene glycol); pegylated phospoholipids such as phophatidylethanolamine-N-[methoxy(polyethyleneglycol)-1000], phophatidylethanolamine-N-[methoxy(polyethyleneglycol)-2000], phophatidylethanolamine-N-[methoxy(polyethylene glycol)-3000], phophatidylethanolamine-N-[methoxy(polyethyleneglycol)-5000]; lyso-phosphatidylcholines, lyso-phosphatidylethanolamines, lyso-phosphatidylglycerols, lyso-phosphatidylserines, ceramides, sphingolipids, e.g., sphingomyelin; phospholipids; glycolipids such as ganglioside GMI; glucolipids; sulphatides; phosphatidic acid, such as di-palmitoyl-glycerophosphatidic acid; palmitic fatty acids; stearic fatty acids; arachidonic fatty acids; lauric fatty acids; myristic fatty acids; lauroleic fatty acids; physeteric fatty acids; myristoleic fatty acids; palmitoleic fatty acids; petroselinic fatty acids; oleic fatty acids; isolauric fatty acids; isomyristic fatty acids; isostearic fatty acids; sterol and sterol derivatives such as cholesterol, cholesterol hemisuccinate, cholesterol sulphate, and cholesteryl-(4-trimethylammonio)-butanoate, ergosterol, lanosterol; poly-oxyethylene fatty acids esters and polyoxyethylene fatty acids alcohols; poly-oxyethylene fatty acids alcohol ethers; polyoxyethylated sorbitan fatty acid esters, glycerol polyethylene glycol oxy-stearate; glycerol polyethylene glycol ricinoleate; ethoxylated soybean sterols; ethoxylated castor oil; polyoxyethylene polyoxypropyl-ene fatty acid polymers; polyoxyethylene fatty acid stearates; di-oleoyl-sn-glycerol; dipalmitoyl-succinyl glycerol; 1,3-dipalmitoyl-2-succinylglycerol; 1-alkyl-2-acyl-phosphatidylcholines such as i-hexadecyl-2-palmitoyl-phosphatidylcholine; 1-alkyl-2-acyl-phosphatidylethanolamines such as 1-hexadecyl-2-palmitoyl-phosphatidylethanolamine; 1-alkyl-2-acyl-phosphatidylserines such as 1-hexadecyl-2-palmitoyl-phosphatidylserine; 1-alkyl-2-acyl-phosphatidylglycerols such as 1-hexadecyl-2-palmitoyl-phosphatidylglycerol; 1-alkyl-2-alkyl-phosphatidylcholines such as 1-hexadecyl-2-hexadecyl-phosphatidylcholine; 1-alkyl-2-alkyl-phosphatidylethanolamines such as 1-hexadecyl-2-hexadecyl-phosphatidylethanolamine; 1-alkyl-2-alkyl-phosphatidylserines such as 1-hexadecyl-2-hexadecyl-phosphatidylserine; 1-alkyl-2-alkyl-phosphatidylglycerols such as 1-hexadecyl̂-hexadecyl-phosphatidylglycerol; N-Succinyl-dioctadecylamine; palmitoylhomocysteine; lauryltrimethylammonium bromide; cetyltrimethyl-ammonium bromide; myristyltrimethylammonium bromide; N-[1,2,3-dioleoyloxy)-propyl]-N,N,Ntrimethylammoniumchloride (DOTMA); 1,2-dioleoyloxy-3 (trimethyl-ammonium)propane (DOTAP); and 1,2-dioleoyl-c-(4′-trimethylammonium)-butanoyl-sn-glycerol (DOTB).

The liposomes contained in the liposomal imaging agents disclosed herein can be untargeted liposomes or targeted liposomes, e.g., liposomes containing one or more targeting moieties or biodistribution modifiers on the surface of the liposomes. A targeting moiety can be any agent that is capable of specifically binding or interacting with a desired target. In one embodiment, a targeting moiety is a ligand. The ligand may preferentially bind to and/or internalize into, a cell in which the liposome-entrapped entity exerts its desired effect (a target cell). A ligand is usually a member of a binding pair where the second member is present on, or in, a target cell(s) or in a tissue comprising the target cell. Examples of suitable ligands include: folic acid, protein, e.g., transferrin, a growth factor, an enzyme, a peptide, a receptor. A targeted liposome wherein a targeting moiety is an antibody or a target antigen-binding fragment thereof (generally an immunoglobulin) is called an “immunoliposome”.

In certain embodiments, the liposomes of the liposomal imaging agents exhibit a transmembrane gradient formed by a gradient-forming agent such as a substituted ammonium compound. Alternate loading modalities are described, e.g., in U.S. Pat. No. 8,147,867. Preferably, the higher concentration of the gradient forming agent is in the interior (inner) space of the liposomes. In addition, a liposome composition disclosed herein can include one or more trans-membrane gradients in addition to the gradient created by the substituted ammonium and/or polyanion disclosed herein. For example, liposomes contained in liposome compositions disclosed herein can additionally or alternately include a transmembrane pH gradient, ion gradient, electro-chemical potential gradient, and/or solubility gradient.

It will be appreciated that when a trapping agent is used, excess gradient forming agent can be removed from the liposomes (e.g., by diafiltration) after the loaded component has been entrapped within the liposome.

### Metal Chelator

The metal chelating moiety of the liposomal imaging agent can be any agent capable of stably chelating a divalent metal cation and being retained in the interior of the liposome. Examples of such metal chelating moieties include the compound 4-DEAP-ATSC:

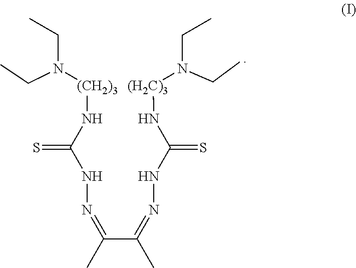

Additional examples of suitable chelators include compounds represented by Formula (IV):

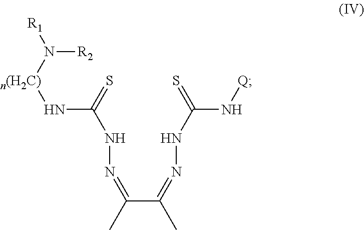

in which

Q is H, substituted or unsubstituted C₁-C₆alkyl or —(CH₂)ₙ—NR₃R₄; R₁, R₂, R₃ and R₄ are each independently selected from H, substituted or unsubstituted C₁-C₆alkyl, or substituted or unsubstituted aryl or wherein either or both of (1) R₁ and R₂ and (2) R₃ and R₄ are joined to form a heterocyclic ring;

and

n is independently, for each occurrence, an integer from 1 to 5.

### Divalent Metal Cation

In some embodiments the metal ion chelated by the chelator is a divalent metal cation. The metal cation for use in the liposomal imaging agents disclosed herein can be any suitable divalent metal cation, e.g., of the alkaline earth, transition metal, lanthanide, or actinide series. A divalent metal cation can be selected according to the intended use of the liposomal imaging agent.

For example, for use in positron emission computed tomography (PET/CT scanning), a positron-emitting radioisotope (such as a divalent ion of ⁴⁴Sc²⁺, ⁶⁴Cu²⁺, ¹¹⁰In²⁺ or ¹²⁸Cs²⁺) can be employed. In certain embodiments, the divalent metal cation is ⁶⁴Cu²⁺. In some embodiments, an x-ray computerized tomography (x-ray CT) scan is performed concomitantly with a PET/CT scan and the images aligned and overlaid upon each other (a PET/x-ray overlay).

### Preparation of Liposomal Imaging Agents

Gradient-based drug loading technologies, in which, e.g., electrochemical gradients drive the accumulation of drugs in the liposome interior, can be used to prepare liposomes. Thus, a liposome having, e.g., an electrochemical gradient between the interior and the exterior of the lipid bilayer can be loaded with cationic chelation complexes of divalent metals by addition of the cationic chelator complex to the liposome preparation.

In general, liposomes can be prepared according to any method known in the art. Other methods for producing nanoparticles/liposomes are disclosed, e.g., in U.S. Patent Application Nos. 20030118636; 20080318325; and 20090186074 and U.S. Pat. Nos. 4,192,869; 4,397,846; 4,394,448; 4,394,149; 4,241,046; 4,598,051; 4,429,008; 4,755,388; 4,911,928; 6,426,086; 6,803,053; 7,871,620; 8,147,867 and 8,329,213.

Alternatively, a liposome can be loaded with an un-complexed chelator moiety (i.e., without a metal cation complexed to the chelator moiety), followed by addition of the divalent metal cation to the liposomal preparation. In one embodiment, the intraliposomal pH is adjusted so that ⁶⁴Cu penetrates the lipid bilayer and forms a complex with the chelator inside the liposome.

### III. Diagnostics and Drug Delivery

The Liposomes disclosed herein may be used for patient stratification or determination of the suitability of a patient for a candidate liposome-based therapy. An exemplary method of determining whether a patient is a candidate for therapy with a liposomal therapeutic agent is as follows:
(a) injecting the patient with a liposomal imaging agent;
(b) imaging the patient to determine the distribution of the liposomal imaging agent within the body of the patient; and
(c) determining that the patient is a candidate for therapy with the liposomal therapeutic agent if the liposomal imaging agent is distributed to a location within the body of the patient in need of the liposomal therapeutic agent.

In another aspect, the invention provides a method of monitoring treatment of a location within the patient by a liposomal therapeutic agent, the method comprising:
(a) injecting the patient with a liposomal imaging agent liposomal imaging agent; and
(b) imaging the patient, wherein a treatment that reduces or eliminates distribution of the liposomal imaging agent to the location within the patient is identified as effective.

In general, the liposomal imaging agents disclosed herein may be used to image a variety of neoplasias including, but not limited to, glioma, astrocytoma, chordoma, craniopharyngioma, acoustic neuroma, medulloblastoma, meningioma, metastatic brain tumors, pituitary tumors, oligodendroglioma, schwannoma, CNS lymphoma, ependymoma, pineal tumors, brain stem glioma, rhabdoid tumors, juvenile pilocytic astrocytoma, primitive neuroectodermal tumors, optic nerve glioma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric carcinoma, gastro-esophageal junction cancer, esophageal cancer, colon carcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, non-small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, lymphoma, oligodenroglioma, schwannoma, meningioma, melanoma, neuroblastoma, and retinoblastoma.

In another embodiment, liposomal imaging agents may be used to image vascular damage caused by a variety of infectious agents including, but not limited to, bacteria, fungi, and viruses. Likewise, the liposomal imaging agents may be used to monitor a patient during treatment for vascular disorders such as hand-foot syndrome (also known as palmar-plantar erythrodysesthesia (PPE), plantar palmar toxicity, palmoplantar keratoderma, and cutaneous toxicity), which is a side effect of some chemotherapy drugs. Hand-foot syndrome results when a small amount of an anti-neoplastic agent leaks out of the smallest blood vessels in the palms of the hands and soles of the feet. The amount of drug in the capillaries of the hands and feet increases due to the friction and subsequent heat that is generated in those extremities. As a result, more drug may leak out of capillaries in these areas. Once out of the blood vessels, the chemotherapy drug damages surrounding tissues. Liposomal imaging agents may be used to image such damage and treatment of the patient can be adjusted accordingly, either by adjusting the dose of drug or by increasing adjunctive therapies such as administration of anti-inflammatory therapeutics. Liposomal imaging agents may also be used to predict those patients who are most likely to experience such side effects and prophylactic adjunctive therapies may be employed.

The quantity of liposome composition necessary to image a target cell or tissue can be determined by routine in vitro and in vivo methods. Safety testing of such compositions will be analogous to those methods common in the art of drug testing. Typically the dosages for a liposome composition disclosed herein ranges between about 0.0007 and about 10 mg of the liposomes per kilogram of body weight. In an exemplary embodiment, the dosage is about 0.0007 mg of the liposomes per kilogram of body weight.

Typically, the liposome pharmaceutical composition disclosed herein is prepared as a topical or an injectable, either as a liquid solution or suspension. However, solid forms suitable for solution in, or suspension in, liquid vehicles prior to injection can also be prepared.

The liposome composition disclosed herein can be administered in any way which is medically acceptable which may depend on the neoplasia being imaged. Possible administration routes include injections, by parenteral routes such as intramuscular, subcutaneous, intravenous, intraarterial, intraperitoneal, intraarticular, intraepidural, intrathecal, or others, as well as oral, nasal, ophthalmic, rectal, vaginal, topical, or pulmonary, e.g., by inhalation. The compositions may also be directly applied to tissue surfaces.

### IV. Study Design

Although ⁶⁴Cu-Liposome B has not been tested in humans, ⁶⁰Cu-ATSM and ⁶⁴Cu-ATSM have been evaluated in human trials as potential imaging agents. The ⁶⁴Cu with 4-DEAP-ATSC used label Liposome B is derived from ⁶⁴Cu-ATSM. In human studies there were no clinically significant changes in vital signs or laboratory test results after injection of ⁶⁰Cu-ATSM and ⁶⁴Cu-ATSM. No adverse events or clinically detectable pharmacologic effects related to either ⁶⁰Cu-ATSM or ⁶⁴Cu-ATSM were observed.

A Parent Study is enrolled that is a Phase 1, multi-center, open-label, dose-escalation, safety, and pharmacokinetic clinical study of intravenously administered Liposome B monotherapy and combination therapy for patients with advanced HER2 positive breast cancer. Disclosed herein are methods and procedures for a Companion Study that will also be enrolled; the Companion Study is an open label, multicenter, single-dose, radiation dosimetry, and biodistribution study of ⁶⁴Cu-Liposome B in patients with advanced cancers. Ten to 45 evaluable patients will be enrolled. Patients will be screened and eligibility confirmed to participate in the Parent Study. A minimum of 6-10 patients is anticipated to obtain sufficient radiation dosimetry assessments. The number of patients may be extended depending on human biodistribution and acquired image quality. After the radiation dosimetry has been evaluated, dosing may be adjusted.

Each patient receives one dose of study treatment (unlabeled Liposome B+⁶⁴Cu-Liposome B), h unlabeled Liposome B, according to the schedule set forth in the Parent Protocol. Participation on this companion protocol will last until all required assessments are completed, approximately 48 hours post-dose. All subsequent study visits and treatment administration will be conducted according to the parent protocol. Dose levels are described below in Table 1.

TABLE 1
Dose levels for Liposome B + ⁶⁴Cu-Liposome B administration

| | | Total Liposome B Dose^{a,c} |
|---|---|---|
| Liposome B | ⁶⁴Cu-Liposome B | (Liposome B + ⁶⁴Cu-Liposome B) |
| (mg/m²) | (mg/m²)^{a} | (mg/m²) |
| 30 | 3-5 | 33-35 |
| 40^{b} | 3-5 | 43-45 |

^{a}The dose range indicated is an approximate dose; the actual dose will depend on the time of administration of ⁶⁴Cu-Liposome B and the patient's body surface area (BSA).
^{b}This dose will only be used if patients are to enroll in a parent protocol cohort using this dose.
^{c}Additional doses may be examined as deemed appropriate by the Investigators, Sponsor, and Medical Monitor.

A variation of plus or minus (+/−) 15% in the dose in millicuries (mCi) of ⁶⁴Cu administered in accordance with this disclosure is provided for in the methods disclosed herein. This is needed because dosage of ⁶⁴Cu (e.g., in mCi/mL) is measured at the radiopharmacy and variability will occur due to alterations such as changes in the timing of the delivery of ⁶⁴Cu preparations from the radiopharmacy to the clinic and in the timing of administration to the patient following delivery, which can significantly alter the dose administered due to the short half-life (about 12.7 hours) of 64Cu. In one embodiment, the about 10.8 mCi is between about 9.72 and about 11.88 mCi. In another embodiment, the about 10.8 mCi is between about 9.18 and about 12.42 mCi.

In some embodiments it is desirable to limit the amount of radioactivity that is administered to a patient. Thus, in some embodiments, a patient may be given a reduced dose of ⁶⁴Cu-Liposome B. As a non-limiting example, a reduced dose maybe used for patients of small stature, for patients who have already recently been exposed to radiation in another capacity, or for patients who are scheduled for an extended imaging time period. Such a reduced dose may comprise, for example, a total of 5 mCi, 7 mCi, or 10 mCi.

By the same token, in cases where a shortened imaging time is necessary, or in cases where increased signal-to-background ratio of the image is desired, the amount of ⁶⁴Cu may be increased up to about 15 mCi, or higher if the radiation dosimetry profile is deemed tolerable. For example, an increased dose might be used if a patient is suspected of having a number of smaller metastatic lesions with low ⁶⁴Cu-Liposome B uptake, or high background tissue signal, making the increased resolution desirable. An increased dose might also be used in the case of late stage cancer patients who are permitted a higher dose of radiation.

The total dose of Liposome B is administered in two stages: (1) unlabeled Liposome B (non-radioactive) followed by (2)⁶⁴Cu-Liposome B (radioactive) up to six hours later. Following administration of ⁶⁴Cu-Liposome B, a transmission scan is acquired using a low-dose CT scan. Patients are imaged in a supine position on a PET/CT scanner in high-sensitivity three-dimensional (3D) mode. Each patient undergoes 2-3 scan sessions at different times, as assigned upon enrollment. After the radiation dosimetry has been evaluated, the number of scans and time points is adjusted if necessary. Subsequent cycles of unlabeled Liposome B will be administered under the parent Protocol. The study schema is outlined below.

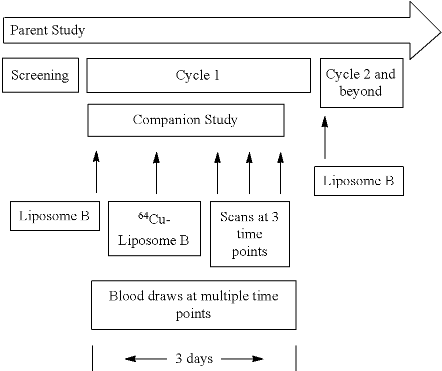

Safety data, including AEs and SAEs, is monitored on an ongoing basis by the study Investigators, the Medical Monitor, and a Sponsor representative as part of routine investigator meetings. Patients are enrolled and dosed according to protocol unless it has been determined that dose-limiting toxicities have occurred in any of the first 6-10 patients. Once 6-10 patients have enrolled, the dosimetry data is reviewed to determine if the dose of ⁶⁴Cu-Liposome B should be adjusted. Any decision or recommendations made during the Investigator meetings is documented in the meeting minutes.

### IV. Pharmacokinetic Assessments

The plasma pharmacokinetic (PK) analyses are performed at the specified times described in Table 2 and Table 3. Blood plasma samples (˜5 mL) is collected and analyzed for unlabeled Liposome B (Table 2). Blood samples taken after ⁶⁴Cu-Liposome B administration are analyzed for radioactivity using a gamma counter (Table 3). The actual time of blood collection must be documented in the respective electronic case report form, and any deviations outside of the time limits must be commented upon. The scheduled blood sampling times are used for the PK analysis; however, any deviations outside the limits (real times) are relevant and the data sets are then adjusted for the PK evaluations and the real times are used.

TABLE 2
PK Sampling Times for unlabeled Liposome B

| Visit | Collection Times |
|---|---|
| Cycle 1, | Pre-dose (within 5 min prior to |
| Day of Liposome B + | dosing with Liposome B) |
| ⁶⁴Cu-Liposome B dosing | Immediately after the end of infusion |
| | of Liposome B (within 5 min) |

TABLE 3
PK Sampling Times for ⁶⁴Cu-Liposome B*

| Visit | Collection Times |
|---|---|
| Cycle 1, | Immediately after the end of infusion of |
| Day of Liposome B + | ⁶⁴Cu-Liposome B (within 1 hr) |
| ⁶⁴Cu-Liposome B dosing | Before or after 1^{st} PET/CT scan (within 1 hr |
| | before or 1 hr after scan is complete) |
| 24 hrs-48 hrs post-dose | Before or after 2^{nd} PET/CT scan (within 1 hr |
| | before or 1 hr after scan is complete) |
| | Optional sample: if 3^{rd}, optional PET/CT |
| | scan is performed, collect another PK |
| | sample within 1 hr before or 1 hr after |
| | scan is complete |

*Samples will be radioactive; appropriate precautions should be taken when processing and handling these samples.

### V. Imaging Procedures

On the day of treatment with ⁶⁴Cu-Liposome B, transmission scans are acquired using a low-dose CT scan. After administration of the ⁶⁴Cu-Liposome B, patients are imaged in a supine position on a PET/CT scanner in high-sensitivity mode. Patients are assigned in an alternating fashion to either early or late scan groups at the time of enrollment, to ensure data are gathered across various time points. Each patient undergoes 2-3 scan sessions at different times as described in Table 4 below. Vital signs are measured and recorded prior to and at the end of each PET/CT scanning procedure.

TABLE 4
PET/CT Imaging Scan Times^{c}

| | Scan | | |
|---|---|---|---|
| Group^{a} | Scan #1 | Scan #2^{b} | Scan #3^{b} |
| 1 | Within 3 hours after | Within 18-30 hours | Within 18-30 hours |
| | ⁶⁴Cu-Liposome B | post-dose of | post-dose, but at |
| | infusion (+3 hrs) | ⁶⁴Cu-Liposome B | least 4 hrs after |
| | | (optional) | Scan #2 |
| 2 | Within 3 hours after | Within 42-54 hours | Within 18-30 hours |
| | ⁶⁴Cu-Liposome B | post-dose of | post-dose of |
| | infusion (+3 hrs) | ⁶⁴Liposome B | ⁶⁴Cu-Liposome B |
| | | | (optional) |

^{a}Each patient is assigned to a scan group at the time of enrollment.
^{b}The 2nd scan is optional for Scan Group 1 and the 3rd scan is optional for Scan Group 2. Once the dosimetry has been determined, the scan time points are adjusted

### EXAMPLES

### Example 1

### ⁶⁴Cu-Liposomes In Vitro and In Vivo Pharmacology

The labeling of liposomes with ⁶⁴Cu is performed using a novel, gradient-loadable chelator named 4-DEAP-ATSC. 4-DEAP-ATSC was derived from ATSM, a copper (Cu) chelator. 4-DEAP-ATSC tightly binds Cu and, by virtue of its amphipathic nature, is able to carry the Cu across liposomal membranes. The manufacturing of, e.g., ⁶⁴Cu-loaded HER2-targeted liposomal doxorubicin (Liposome B) involves the generation of a trans-liposomal membrane pH gradient that is used to load doxorubicin into the acidic interior of the liposomes. Following manufacturing, there is a residual gradient remaining that can be used to load 4-DEAP-ATSC (and its complex with Cu) into HER2-targeted liposomal doxorubicin. Once inside the liposomes, 4-DEAP-ATSC is believed to become protonated, which then restricts its ability to cross the liposomal membrane, resulting in entrapment of ⁶⁴Cu in the interior of the liposome. Loading of copper into liposomes is described in detail in, e.g, co-pending patent application PCT/US13/37033.

### Example 2

### In Vitro Stability of ⁶⁴Cu:4-DEAP-ATSC-Loaded Liposomes in Human Plasma

⁶⁴Cu:4-DEAP-ATSC has been successfully loaded into liposomal formulations that contain chemotherapeutic agents via the residual chemical gradient. Examples of such liposomal formulations include the HER2-targeted doxorubicin-loaded Liposome B, the irinotecan-loaded Liposome C, as well as the commercially available doxorubicin-loaded Doxil®. ⁶⁴Cu:4-DEAP-ATSC with chelation efficiency >90% was mixed with varying amounts of Liposome B, Liposome C, or Doxil®. The mixture was then incubated in a water bath at 65° C. for 10 minutes and the loading procedure was subsequently quenched in an ice water bath. Using size exclusion chromatography, it was determined that more than 90% of ⁶⁴Cu:4-DEAP-ATSC can be loaded into Liposome B (Table 5), Liposome C (Table 6), and Doxil® (Table 7) below.

TABLE 5
⁶⁴Cu-loaded Liposome B

| 4-DEAP- | ⁶⁴Cu |
|---|---|
| ATSC:Doxorubicin | Loading |
| Ratio (mol %) | Efficiency |
| 0.16 mol % | 98% |
| 0.7 mol % | 95% |
| 1.0 mol % | 92% |
| 1.6 mol % | 95% |
| 2.0 mol % | 92% |
| 2.7 mol % | 96% |
| 4.0 mol % | 93% |
| 8.0 mol % | 93% |
| 40 mol % | 90% |

TABLE 6
⁶⁴Cu-loaded Liposome C

| 4-DEAP- | ⁶⁴Cu |
|---|---|
| ATSC:Irinotecan | Loading |
| Ratio (mol %) | Efficiency |
| 0.01 mol % | 97% |
| 0.2 mol % | 95% |
| 0.6 mol % | 97% |
| 2.5 mol % | 97% |
| 12.5 mol % | 90% |

TABLE 7
⁶⁴Cu-loaded Doxil ®

| 4-DEAP- | ⁶⁴Cu |
|---|---|
| ATSC:Doxorubicin | Loading |
| Ratio (mol %) | Efficiency |
| 0.6 mol % | 94% |
| 2.0 mol % | 96% |
| 4.0 mol % | 96% |
| 8.0 mol % | 96% |
| 40 mol % | 91% |

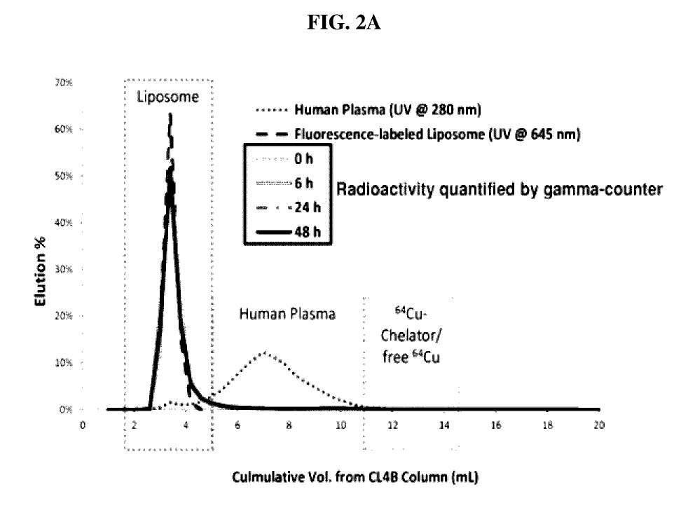

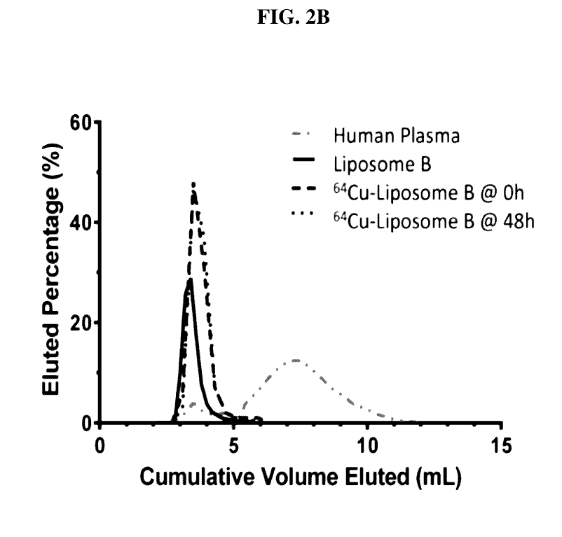

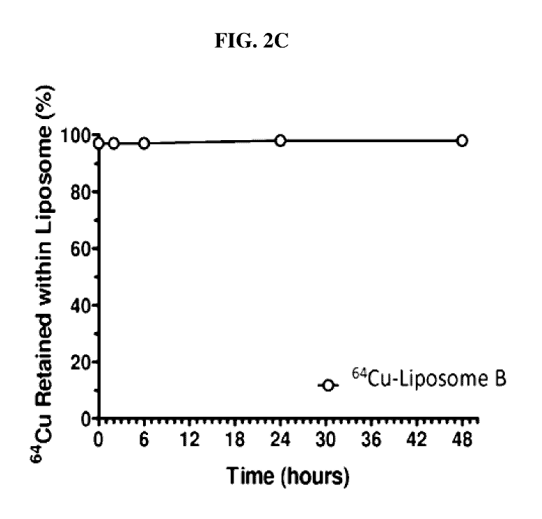

⁶⁴Cu was shown to be effectively retained in the liposome after incubation of ⁶⁴Cu:4-DEAP-ATSC-loaded liposome (Liposome A) in human plasma for 48 hours (FIG. 2A). The in vitro stability of Liposome A was examined by incubating the ⁶⁴Cu:4-DEAP-ATSC-loaded liposome with human plasma at 37° C. At the designated incubation time (up to 48 hours), encapsulated (liposomal) radioactivity was separated from released/unencapsulated radioactivity using size exclusion chromatography (CL-4B SEC column, which separates liposomal, protein, and ⁶⁴Cu:4-DEAP-ATSC/uncomplexed ⁶⁴Cu fractions). In FIG. 2A, all of the radioactivity at each time point indicated in the inset (0, 6, 24 and 48 hours) falls on the same curve, thus appearing as a solid line peaking between 2 and 4 on the X axis. This peak shows the % of radioactivity in each elution fraction, not the total amount, thus there is no differential resulting from radioactive decay, and the lack of any differential due to release from liposomal entrapment is evidenced by the single peak within the volume range where liposomes elute from the size exclusion chromatography column, and not in volume ranges where plasma or free copper elute (as indicated by the shaded boxes). The data show that Liposome A is highly stable in human plasma at physiological temperature, with <5% of unencapsulated ⁶⁴Cu detected up to 48 hours.

The stability of the ⁶⁴Cu-Liposome B was evaluated in vitro by incubation of ⁶⁴Cu-Liposome B in human plasma at 37° C. for up to 48 hours. Size exclusion chromatography was then performed to separate liposomal ⁶⁴Cu from free ⁶⁴Cu, and radioactivity was quantified by gamma counter, shown in FIG. 2B. Greater than 95% of ⁶⁴Cu was in the liposomal fraction immediately after loading, illustrating >95% loading efficiency. After 48 hours of incubation in human plasma, >95% of ⁶⁴Cu remained encapsulated in liposomes, shown in FIG. 2C. This demonstrates that ⁶⁴Cu-Liposome B stably retains the ⁶⁴Cu label over the timeframe that patients will be imaged by PET.

### Example 3

### Pharmacokinetics and In Vivo Stability of ⁶⁴Cu-Liposome B

Naïve CD-1 mice were injected with ⁶⁴Cu-Liposome B, free ⁶⁴Cu or ⁶⁴Cu:4-DEAP-ATSC complex. Plasma samples were collected via saphenous vein puncture at designated time points. The ⁶⁴Cu and doxorubicin contents in the plasma were analyzed via gamma-counting or HPLC, respectively. All data are decay-corrected to the injection time. B, the ratio of ⁶⁴Cu to doxorubicin was calculated from the ⁶⁴Cu-Liposome B data in A.

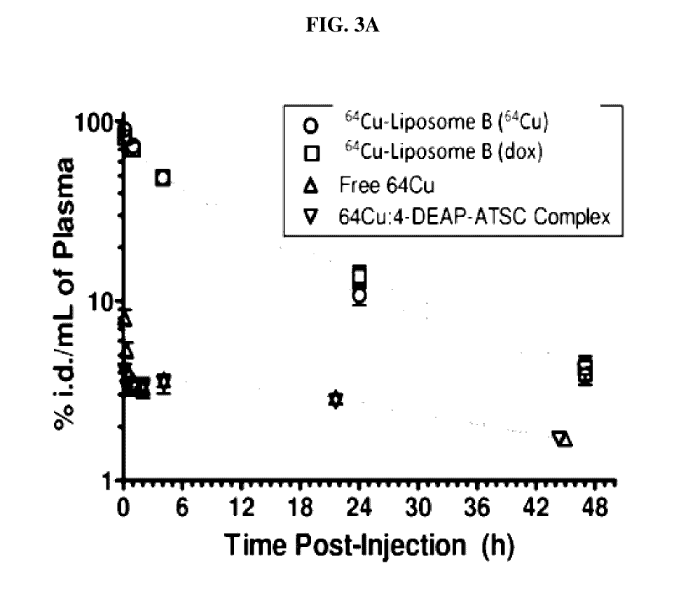

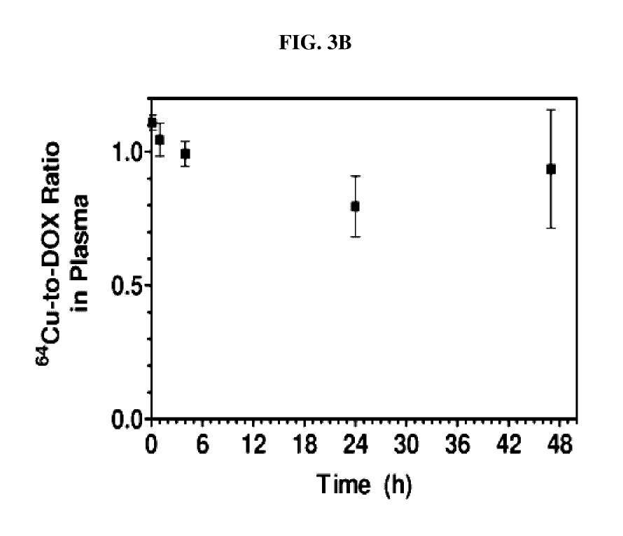

The pharmacokinetics of ⁶⁴Cu-Liposome B was evaluated in non-tumor bearing CD-1 mice, and was assessed by measuring both ⁶⁴Cu and doxorubicin in plasma samples, as shown in FIG. 3A. The stability of the ⁶⁴Cu label is demonstrated by comparing ⁶⁴Cu to doxorubicin over time, shown in FIG. 3B, indicating that approximately 90% of the ⁶⁴Cu is stably retained within the liposomes. For comparison, the pharmacokinetics of free ⁶⁴Cu and the ⁶⁴Cu:4-DEAP-ATSC complex were also studied and both show a very rapid initial clearance followed by a slow elimination phase (FIG. 3A).

### Example 4

### Biodistribution of ⁶⁴Cu-Liposome B

A biodistribution study was performed in BT-474-M3 xenograft tumor-bearing mice to determine the correlation between ⁶⁴Cu levels and doxorubicin levels in the tumor and other tissues following dosing with ⁶⁴Cu-Liposome B. Mice (n=4) were dosed with 3 mg/kg of ⁶⁴Cu-Liposome B by tail vein injection. Twenty-four hours post-injection, mice were perfused with 20 mL phosphate-buffered saline and tissues harvested. ⁶⁴Cu content was measured by gamma-counter and doxorubicin content measured by HPLC, correcting for extraction efficiency. ⁶⁴Cu data are decay-corrected to the time of injection. *p<0.01. Similar values of ⁶⁴Cu and doxorubicin were measured in the tumor, as shown in FIG. 4, suggesting that measurement of ⁶⁴Cu levels in the tumor by PET provides an accurate assessment of the amount of Liposome B deposition in tumors. Similar results were also determined in the spleen and liver, suggesting that ⁶⁴Cu provides an accurate assessment of the amount of Liposome B distributed to those tissues.

### Example 5

### Effect of Liposome Targeting on Tumor Deposition

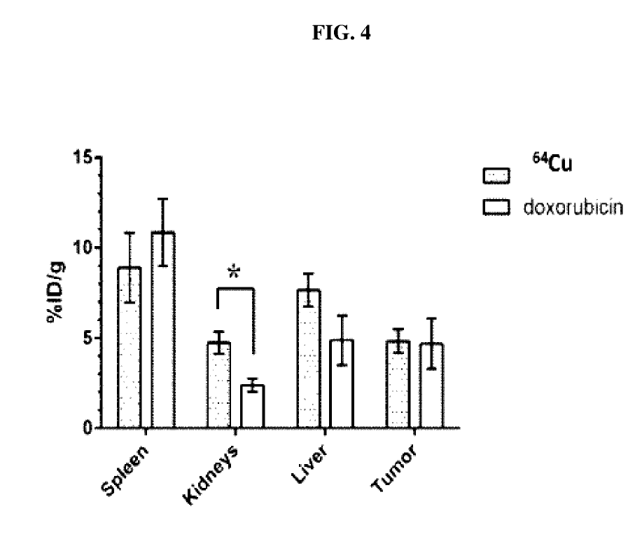

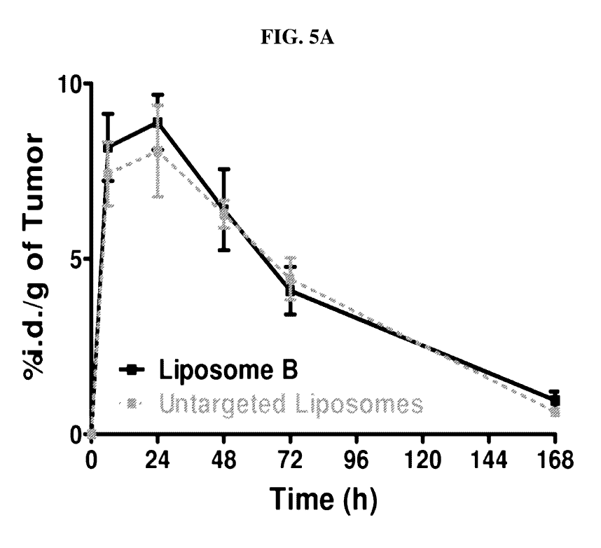

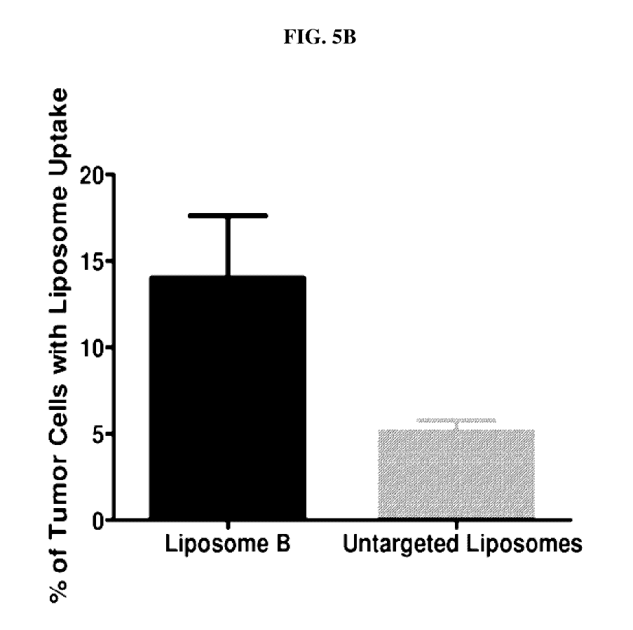

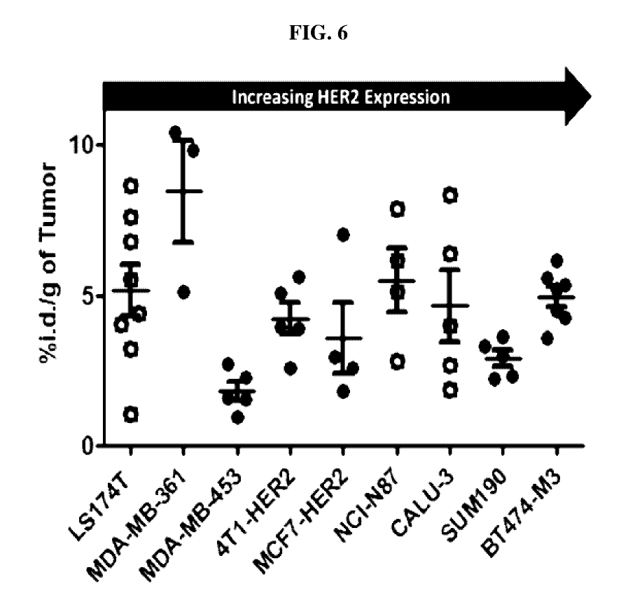

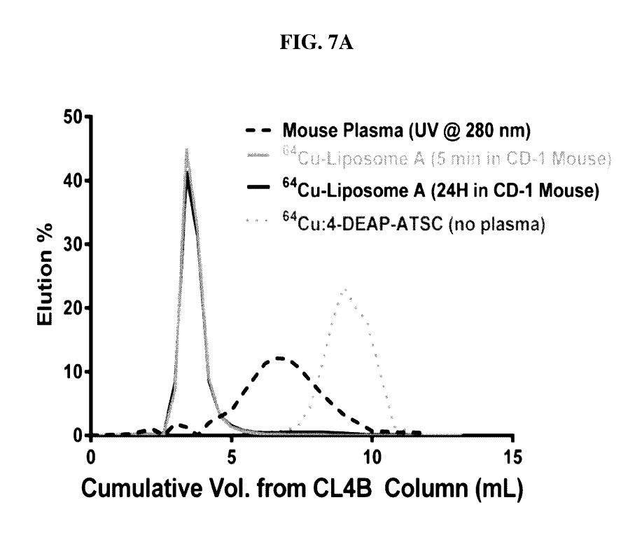

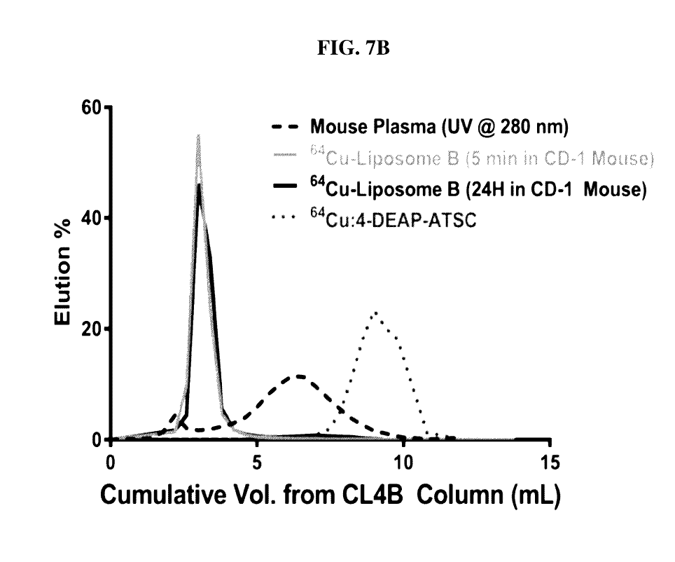

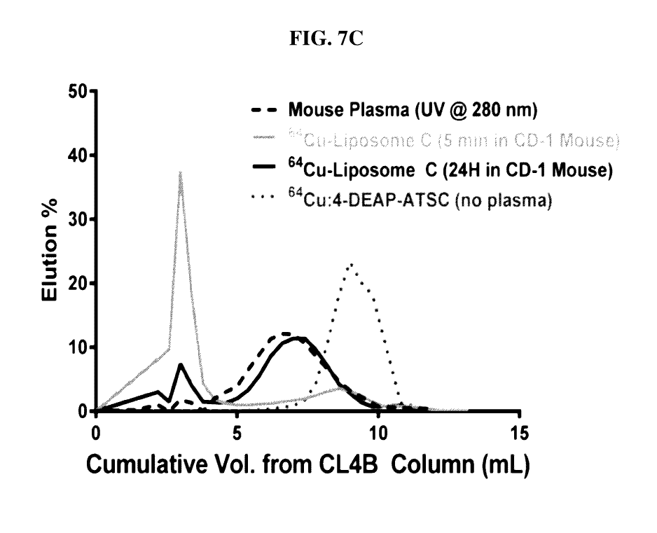

Preclinical studies have examined the effect of liposome targeting on total tumor deposition. These studies have shown that the targeting of PEGylated liposomes to the HER2 receptor on tumors did not affect its pharmacokinetics or overall tumor deposition compared to an untargeted liposome. Kirpotin et al labeled liposomes with ⁶⁷Ga and showed similar tumor deposition % injected dose per gram (% i.d./g) for a HER2-targeted liposome and a corresponding untargeted liposome (Cancer Research (66)6732 (2006). Similar results were obtained by comparing tumor deposition by HER2-targeted Liposome B and untargeted liposomes (disclosed in co-pending Patent Application Serial No. PCT/US2011/064496) in an NCI-N87 (ATCC® #CRL-5822™) gastric carcinoma mouse xenograft model, as well as in BT474-M3 breast carcinoma mouse xenograft model in which the two liposome formulations only result in difference in tumor cell uptake (FIG. 5 insert) with no significant difference detected in total liposome deposition in the tumors (FIG. 5). FIG. 6 further illustrates that liposome targeting does not have any obvious effect on tumor deposition as no correlation can be established between tumor depositions of Liposome B in tumors with varying HER2 expression. Similarly, in the BT474-M3 tumor model (HER2-overexpressing tumors), the HER2-targeted liposome B were shown to have similar tumor deposition as the non-targeted liposome A.

### Example 6

### PET/CT Imaging of ⁶⁴Cu-Liposome B in Mice

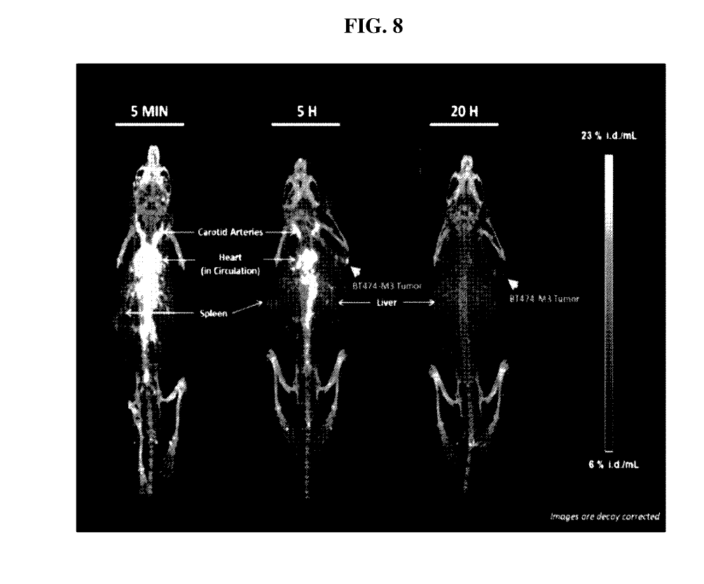

PET/CT imaging was performed in BT-474-M3 tumor bearing mice injected intravenously with ⁶⁴Cu-Liposome B. ⁶⁴Cu-Liposome B accumulated mainly in the liver and spleen, as well as in circulation as a result of the long-circulating characteristics of the disclosed liposomes (FIG. 8). Significant accumulation of ⁶⁴Cu-Liposome B was also detected at the tumor site at 5 and 20 hours post-injection.

### Example 7

### PET/CT Imaging of ⁶⁴Cu-Liposome B in Humans

The dosimetry of ⁶⁴Cu-Liposome B at the organ level was studied in the mouse using standard methods and predicted human radiation absorbed doses to the kidneys, liver and spleen of 0.083 mGy/MBq (0.307 (rad/mCi)), 0.069 (0.256) and 0.06 (0.220), respectively. At the whole organ level, it is predicted that the kidney will be the dose-limiting organ.

The proposed starting radiation dose of ⁶⁴Cu-Liposome B for humans is 400 MBq (with a range of 320-440 MBq). The radiation dose may be adjusted after obtaining improved estimates of dosimetry in humans. Based on preclinical dosimetry estimates in mice, the predicted radiation absorbed doses to the kidneys, liver and spleen are 33.2, 27.6 and 24 mGy, respectively. These values are consistent with radiation absorbed doses observed in other clinical studies with ⁶⁴Cu-labeled agents and with radiolabeled liposomes.

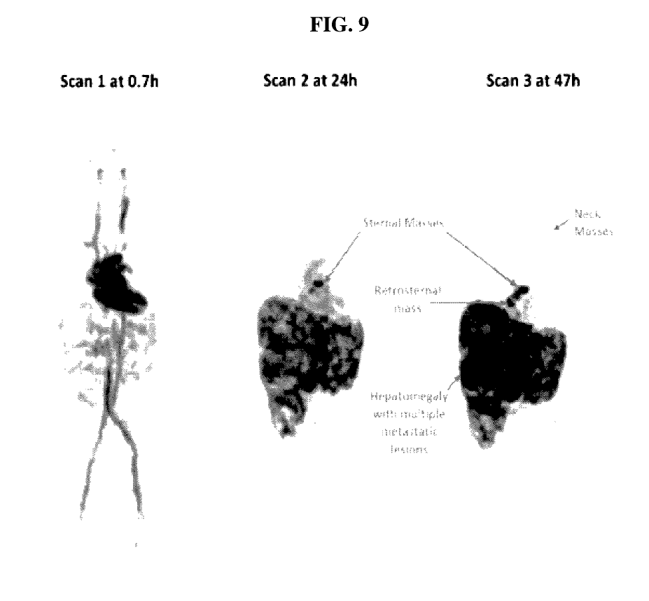

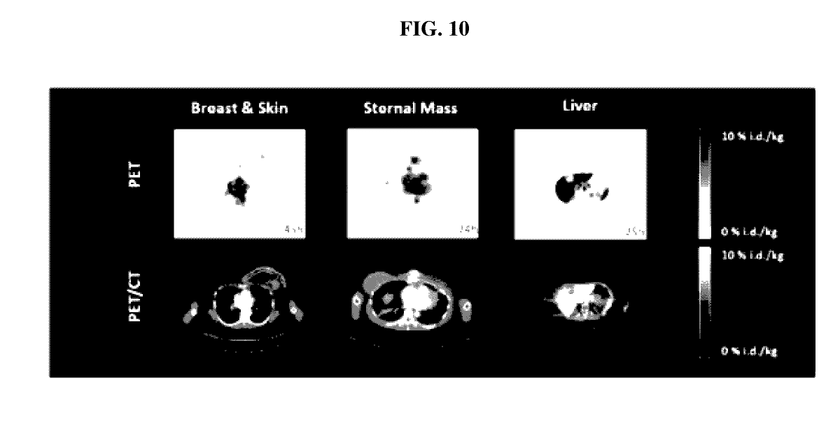

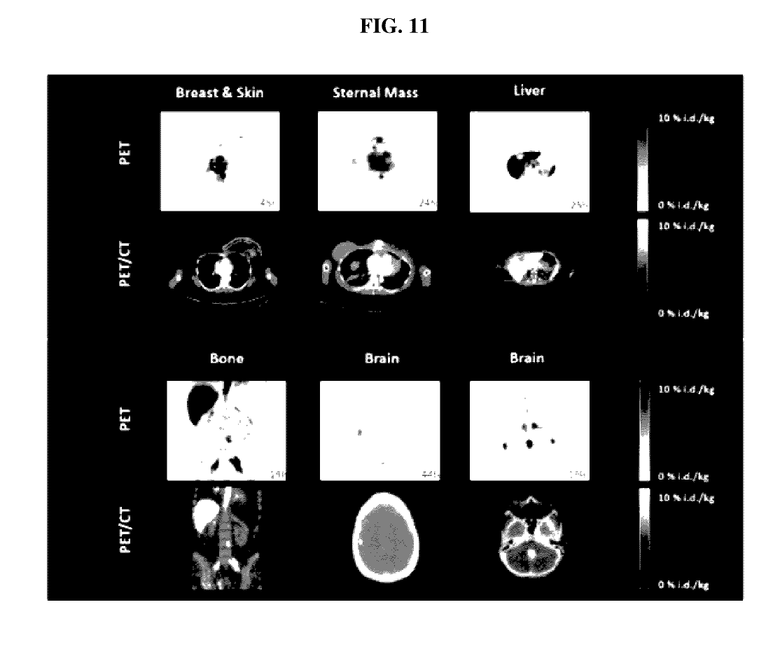

PET/CT imaging was performed on human cancer patients after administration of ⁶⁴Cu-Liposome B at a dose of approximately 400 MBq. Radiation dosimetry from 11 patients was estimated to result in radiation absorbed doses to the kidneys, liver, and spleen at 8.0, 46.4, and 59.6 mGy. 400 MBq of administered ⁶⁴Cu-Liposome B was able to provide adequate PET image quality for quantification assessment from <3 h to at least 48 h post-injection. As can be seen from the images in FIGS. 9-11, the ⁶⁴Cu-loaded liposomes accumulated preferentially in a variety of metastatic lesions, including liver (FIG. 9), bone (FIG. 10), and brain (FIG. 11) as well as breast, skin, sternum, and neck, while blood signal decreased over time.

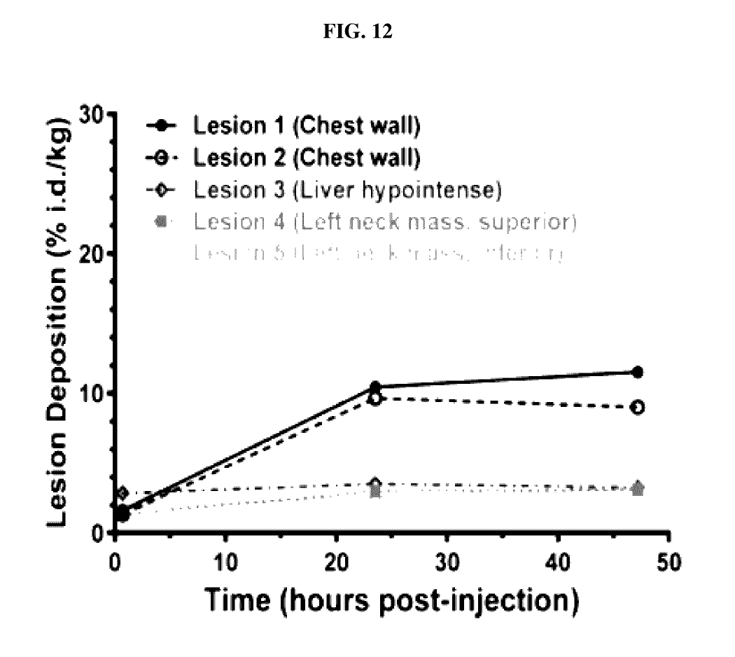

FIG. 12 is a graph showing examples of ⁶⁴Cu-liposome B deposition kinetics in 5 lesions in a single patient within 48 hours post-injection of the liposome. PET/CT images were acquired at 0.7, 24, and 47 hours post-injection.

Endnotes

While the invention has been described in connection with specific embodiments, it will be understood that it is capable of further modifications and this application is intended to cover any variations, uses, or adaptations including such departures from the present disclosure that come within known or customary practice within the art. Any combination or combinations of each of the embodiments disclosed in the dependent claims is contemplated within the scope of this disclosure. The disclosure of each and every U.S., international, or other patent or patent application or publication referred to herein is hereby incorporated herein by reference in its entirety for all purposes.

What is claimed is:

1. A composition comprising ⁶⁴Cu-loaded HER2-targeted immunoliposomes containing doxorubicin, wherein the ⁶⁴Cu-loaded HER2-targeted immunoliposomes comprise hydrogenated soy phosphatidylcholine (HSPC), cholesterol, and poly(ethylene glycol) (PEG)-derivatized distearoylphosphatidylethanolamine (PEG-DSPE) at a 3:1:0.05 molar ratio.

2. The composition of claim 1, wherein the composition is adapted for administration to a human patient at a dose of at least 0.028, at least 3, at least 4, at least 5, at least 6, or 7 mg/m² of doxorubicin.

3. The composition of claim 1, wherein the ⁶⁴Cu-loaded HER2-targeted immunoliposomes comprise a gradient-loadable chelator.

4. The composition of claim 3, wherein the chelator is 4-DEAP-ATSC.

5. The composition of claim 1, wherein the composition comprises at least 1, at least 5, at least 10, about 10.8, about 12, or about 15 mCi of ⁶⁴Cu.

6. A method of imaging a lesion in a patient, the method comprising:
(a) administering to the patient an injection comprising a preparation of ⁶⁴Cu-loaded HER2-targeted immunoliposomes, the immunoliposomes having an average diameter of 75-110 nm, and a dose of 3-5 mg/m² doxorubicin;
(b) obtaining a PET scan of a region of the patient, the region comprising the location of the lesion within 48 hours following the injection.

7. The method of claim 6, wherein the starting dose of ⁶⁴Cu-loaded HER2-targeted immunoliposomes ranges from 320-440 MBq.

8. The method of claim 6, wherein the ⁶⁴Cu-loaded HER2-targeted immunoliposomes comprise a gradient-loadable chelator.

9. The method of claim 8, wherein the chelator is 4-DEAP-ATSC.

10. The method of claim 6, wherein the lesion is a benign tumor or a malignant tumor.

11. The method of claim 10, wherein the lesion is a malignant brain tumor.

12. The method of claim 6, wherein the dose of ⁶⁴Cu-loaded HER2-targeted immunoliposomes is formulated to deliver to the patient, when administered, 10.8 (+/−15%) mCi of ⁶⁴Cu.

13. The method of claim 6, wherein the PET scan is obtained within 24 hours, within 12 hours, within six hours, within 3 hours, within 2 hours, or within 1 hour following the injection.

14. A method of treating and imaging a patient, the method comprising:
(a) administering to the patient a first injection comprising HER2-targeted immunoliposomal doxorubicin that does not comprise detectable levels of ⁶⁴Cu, the first injection administered at a dose of 30 mg/m² of doxorubicin (doxorubicin HCl equivalent) in a doxorubicin encapsulated HER2-targeted immunoliposome;
(b) at between one and 6 hours following the first injection, administering to the patient a second injection comprising ⁶⁴Cu-loaded HER2-targeted immunoliposomal doxorubicin, containing a dose of doxorubicin of at least 3, at least 4, at least 5, at least 6, or 7 mg/m², said second injection comprising between 9.18 mCi and 12.42 mCi of ⁶⁴Cu; and
(c) obtaining at least two scans selected from the group consisting of PET scans, CT scans or a combination thereof, of a region of pathology in the patient, wherein each scan is obtained at a different time point, and wherein time elapsed from the injection of (a) until a final scan of the at least two scans is obtained is no more than three days.

15. The method of claim 14, wherein the region of pathology is a malignant brain tumor.

16. The method of claim 14, wherein the region of pathology is a metastatic lesion selected from the group consisting of: a liver metastatic lesion, a bone metastatic lesion, a brain metastatic lesion, a breast metastatic lesion, a skin metastatic lesion, and a sternum metastatic lesion.

17. The method of claim 14, wherein the ⁶⁴Cu-loaded HER2-targeted immunoliposomal doxorubicin comprises a gradient-loadable 4-DEAP-ATSC chelator.

18. The method of claim 14, wherein the first scan is obtained within 3 hours following the second injection.

19. The method of claim 18, wherein the first scan is obtained within 2 hours or within 1 hour following the second injection.

20. The method of claim 14, wherein the first scan is obtained within 3 hours following the second injection, a second scan is obtained within 18-30 hours following the second injection and a third scan is obtained 18-30 hours following the second injection, but at least 4 hours after the second scan.

21. The method of claim 14, wherein the first scan is obtained within 3 hours following the second injection, a second scan is obtained within 42-54 hours following the second injection and an optional third scan is obtained 18-30 hours following the second injection.

22. A method of treating and imaging a patient, the method comprising:
(a) administering to the patient once on day 1 of a first 21-day treatment cycle: a first injection comprising an amount of doxorubicin contained in 30 mg/m² of doxorubicin HCl encapsulated in a MM-302 HER2-targeted doxorubicin immunoliposome that does not comprise detectable levels of ⁶⁴Cu;
(b) at between one and 6 hours following the first injection, administering to the patient a second injection comprising ⁶⁴Cu-loaded HER2-targeted immunoliposomal doxorubicin, containing a dose of doxorubicin of at least 3, at least 4, at least 5, at least 6, or 7 mg/m², said second injection comprising between 9.18 mCi and 12.42 mCi of ⁶⁴Cu; and
(c) obtaining at least two scans selected from the group consisting of PET scans, CT scans or a combination thereof, of a region of pathology in the patient, wherein each scan is obtained at a different time point, and wherein time elapsed from the injection of (a) until a final scan of the at least two scans is obtained is no more than three days.
